(19) United States
(12) Patent Application Publication
(10) Pub. No.: US 2008/0082003 A1
(43) Pub. Date: Apr. 3, 2008

(54) Method and apparatus for detecting respiratory rate

(75) Inventors: Yun Deng, Shenzhen (CN); Jilun Ye, Shenzhen (CN)

Correspondence Address: PILLSBURY WINTHROP SHAW PITTMAN LLP, P.O BOX 10500, McLean, VA, 22102, US

(73) Assignee: SHENZHEN MINDRAY BIO-MEDICAL ELECTRONICS CO., LTD., Shenzhen (CN)

(21) Appl. No.: 11/607,605

(22) Filed: Dec. 1, 2006

(30) Foreign Application Priority Data
Aug. 28, 2006 (CN) 200610062356.3

(51) Int. Cl. A61B 5/02; A61B 5/08

(52) U.S. Cl. 600/484; 600/529

(57) ABSTRACT

The invention provides a method and apparatus for detecting respiratory rate, comprising the steps of: sampling a set of respiratory rate data in a time sequence; determining the variation degree for the set of respiratory rate data; determining the base length according to the variation degree for the set of respiratory rate data; calculating the weighted average length according to the base length; calculating the real-time degree for each of the set of respiratory rate data according to the variation degree for the set of respiratory rate data, the sequence number indicating the position for each of the set of respiratory rate data and the weighted average length, the sequence number indicating the position for a preceding respiratory rate data is less than the sequence number indicating the position for a following respiratory rate data; and calculating the weighted average respiratory rate according to the weighted average length and the real-time degree. According to the invention, the calculated respiratory rate data is advantageous in being both real time degree and smoothness of respiratory rate.

### FIELD OF THE INVENTION

The invention relates generally to a respiration monitoring system, and more particularly, to method and apparatus for detecting respiratory rate in the respiration monitoring system.

### BACKGROUND OF THE INVENTION

In a conventional respiration monitoring system, the respiratory rate is generally calculated by following steps: recording a plurality of recent respiratory rate data into an array, refreshing the record of the plurality of respiratory rate data in a FIFO (First in First Out) manner during calculation, and averaging a number of respiratory rate data occurring in a predetermined time period.

First, the number of data to be used in averaging the respiratory rate data is determined as follows:

1. When power on, the number of respiratory rate data increases from 1 to 12 in a sequential order, as determined by MIN (the number of data, 35/the current respiratory period+1); and

2. A period of time after power on, the number of respiratory rate data has exceeded 12, as determined by MIN (12, 35/the current respiratory period+1).

Then, the respiratory rate data is averaged as follows:

1. When the respiratory period is normal (the respiratory rate>=10 BPM), there are at least 6 respiratory rate data in the predetermined time period. At this time, a search is made for the maximum value, the minimum value and the next maximum value in these respiratory rate data, and the average respiratory rate data is equal to the average of the respiratory rate data excluding the three values;

2. When the respiratory period is long (the respiratory rate is low), there will be less than 6 respiratory rate data in the predetermined time period, and the respiratory rate data is averaged in two ways:

2a). When the number of these respiratory rate data>4, the average of these respiratory rate data is equal to the average of these respiratory rate data in the predetermined time period;

2b). When the number of these respiratory rate data≦4, the average of the respiratory rate data is equal to the average of these respiratory rate data excluding one of the maximum value and the minimum value which is more different from the last respiratory rate data value than another.

The respiratory impedance of a patient is prone to exterior interferences, such as movements, heartbeat and so on, causing sudden and frequent changes in the respiratory rate data detected at the respiration monitoring system, which occurs often in the case of monitoring the respiration of a newly-born baby. Sometimes, the respiratory frequency of a patient changes from low to high (or from high to low) very quickly and then enters into a relatively stable respiration state, and it is generally desired that the respiratory rate data detected at the respiration monitoring system can reflect the changing trend of the patient's respiration in a timely manner. Conventional methods for average calculation of the respiratory rate data cannot meet such requirements satisfactorily, and the specific problems are as follows.

1. The calculation result cannot reflect the real-time conditions, because an arithmetic averaging is done only on the real-time respiratory rate data within several time periods, without considering real-time changes in the respiratory rate data; and

2. In conventional methods for average calculation of the respiratory rate data, the averaging of the respiratory rate data usually uses the real-time respiratory rate data in the last 12 periods, without full consideration of influences from the respiratory rate data on the calculation result, which causes poor smoothness in the calculation result, for example, sudden changes are easy to occur in the current respiratory rate data when the real-time respiratory waveform is interfered.

### SUMMARY OF THE INVENTION

To solve the above problems, the invention provides a method and apparatus for detecting respiratory rate, which may produce better real-time performance and smoothness for the respiratory rate data detected at a respiration monitoring system, so as to meet clinical requirements.

In one aspect of the invention, a method for detecting respiratory rate is provided, the method comprising the steps as follows:

A1. sampling a set of respiratory rate data in a time sequence;

B1. determining the variation degree for the set of respiratory rate data;

C1. determining the base length for the set of respiratory rate data according to the variation degree for the set of respiratory rate data;

D1. calculating the weighted average length for the set of respiratory rate data according to the base length for the set of respiratory rate data;

E1. calculating the real-time degree for each of the set of respiratory rate data according to the variation degree for the set of respiratory rate data, the sequence number indicating the position for each of the set of respiratory rate data and the weighted average length for the set of respiratory rate data, the sequence number indicating the position for a preceding respiratory rate data is less than the sequence number indicating the position for a subsequent respiratory rate data; and

F1. calculating the weighted average respiratory rate according to the weighted average length and the real-time degree.

Preferably, the step B1 comprises the steps of:

B11. calculating the variation coefficient between two adjacent respiratory rate data in the set of respiratory rate data;

B12. counting the numbers lcnt, mcnt and hcnt of respiratory rate data whose variation coefficients fall within intervals [0, lcv], [0, mcv] and [0, hcv], respectively, where lcv is a lower critical value, mcv is an intermediate critical value, hcv is an upper critical value (the maximum variation coefficient) and 0=<lcv<mcv<hcv;

B13. determining whether lcnt/N is more than or equal to a first threshold, if yes, the variation degree for the set of respiratory rate data is low, if no, the step B14 is performed, where N is the total number of respiratory rate data in the set;

B14. determining whether mcnt/N is more than or equal to the first threshold. If yes, the variation degree for the set of respiratory rate data is intermediate, if no, the step B15 is performed; and

B15. determining that the variation degree for the set of respiratory rate data is high.

Preferably, the variation coefficient between two adjacent respiratory rate data is cv=(d1−d2)/((d1+d2)/2) if the values for the two adjacent respiratory rate data in the set of respiratory rate data are d1 and d2, respectively, where d0>=0, d2>=0 and d1 or d2 is not equal to 0.

Preferably, the first threshold may be set to 70%, 80% or 90%.

Preferably, the step C1 comprises the steps of:

C11. detecting the variation degree for the set of respiratory rate data; and

C12. determining the base length for the set of respiratory rate data according to the detected variation degree for the set of respiratory rate data, wherein the base length is equal to the number of reference respiratory rates in a predetermined first time period or the number of predetermined first respiratory rates, whichever is larger, when the variation degree for the set of respiratory rate data is low, the number of the predetermined first respiratory rates being the minimum empirical value of respiratory rates in the first time period; the base length is equal to the number of reference respiratory rates in a predetermined second time period or the number of predetermined second respiratory rates, whichever is larger, when the variation degree for the set of respiratory rate data is intermediate, the number of the predetermined second respiratory rates being the minimum empirical value of respiratory rates in the second time period; and the base length is equal to the number of reference respiratory rates in a predetermined third time period or the number of predetermined third respiratory rates, whichever is larger, when the variation degree for the set of respiratory rate data is high, the number of the predetermined third respiratory rates being the minimum empirical value of respiratory rates in the third time period; and wherein the reference respiratory rates represent the closest respiratory rates at present, the first time period<the second time period<the third time period, and the number of the first respiratory rates<the number of the second respiratory rates<the number of the third respiratory rates.

Preferably, the predetermined first time period may last 10 seconds, the number of the predetermined first respiratory rates may be 5, the predetermined second time period may last 15 seconds, the number of the predetermined second respiratory rates may be 6, the predetermined third time period may last 20 seconds, and the number of the predetermined third respiratory rates may be 7.

Preferably, the weighted average length is calculated by the following equation at the step D1,

$$\frac{blrt}{slrt} = \frac{{\sum }_{t = sl - bl + 1}^{sl} {i}^{n}}{{\sum }_{i = 1}^{sl} {i}^{n}} >= 90 \%$$

where i is the sequence number indicating the position for a data, the sequence number indicating the position for a preceding respiratory rate data is less than the sequence number indicating the position for a subsequent respiratory rate data, n is a weighting factor and n≧0, b1 represents the base length, s1 represents the weighted average length, which is the least integer satisfying the equation.

Preferably, the weighting factor n=0 when the variation degree for the set of respiratory rate data is high, the weighting factor n=1 when the variation degree for the set of respiratory rate data is intermediate, and the weighting factor n=2 when the variation degree for the set of respiratory rate data is low.

Preferably, the step E1 comprises the steps of:

E11. detecting the variation degree for the set of respiratory rate data;

E12. selecting a weighting factor based on the variation degree for the set of respiratory rate data, a higher variation degree leading to a smaller weighting factor; and

E13. calculating the real-time degree for each of the set of respiratory rate data according to

$$rt ( i ) = \frac{{i}^{n}}{{\sum }_{i = 1}^{i} {i}^{n}} ,$$

where i is the sequence number indicating the position for a data, the sequence number indicating the position for a preceding respiratory rate data is less than the sequence number indicating the position for a subsequent respiratory rate data, n is a weighting factor and n≧0, l represents the weighted average length for the data used, and rt represents the real-time degree.

Preferably, the weighting factor n=0 when the variation degree for the set of respiratory rate data is high; the weighting factor n=1 when the variation degree for the set of respiratory rate data is intermediate; and the weighting factor n=2 when the variation degree for the set of respiratory rate data is low.

Preferably, the weighted average respiratory rate is calculated at the step F1 by

$$rtav = {\sum }_{i = 1}^{t = sl} rr ( i ) * \frac{{i}^{n}}{{\sum }_{t = 1}^{sl} {i}^{n}}$$

where rtav represents the weighted average respiratory rate, i is the sequence number indicating the position for a data, the sequence number indicating the position for a preceding respiratory rate data is less than the sequence number indicating the position for a subsequent respiratory rate data, sl represents the weighted average length, n is a weighting factor and n≧0, and rr(i) represents the respiratory rate for the i^{th} data in the set of respiratory rate data.

In another aspect of the invention, an apparatus for detecting respiratory rate is provided, the apparatus comprising:

a sampling module, for sampling a set of respiratory rate data in a time sequence;

a variation degree calculation module, for receiving the set of respiratory rate data from the sampling module and determining the variation degree for the set of respiratory rate data;

a base-length calculation module, for calculating the base length for the set of respiratory rate data according to the determined variation degree for is the set of respiratory rate data;

a weighted average length calculation module, for calculating the weighted average length for the set of respiratory rate data according to the determined base length for the set of respiratory rate data;

a real-time degree calculation module, for calculating the real-time degree for each of the set of respiratory rate data according to the variation degree for the set of respiratory rate data from the variation degree calculation module, the sequence number indicating the position for each of the set of respiratory rate data and the weighted average length for the set of respiratory rate data from the weighted average length calculation module, the sequence number indicating the position for a preceding respiratory rate data is less than the sequence number indicating the position for a subsequent respiratory rate data; and

a weighted average respiratory rate calculation module, for calculating the weighted average respiratory rate for the set of respiratory rate data according to the calculated weighted average length and the calculated real-time degree.

Preferably, the variation degree calculation module comprises: a variation coefficient calculation unit, for receiving the set of respiratory rate data from the sampling module and calculating the variation coefficient between two adjacent respiratory rate data in the set of respiratory rate data; a counting unit, for counting the numbers lcnt, mcnt and hcnt of respiratory rate data whose variation coefficients fall within intervals [0, lcv], [0, mcv] and [0, hcv] respectively, where lcv is a lower critical value, mcv is an intermediate critical value, hcv is an upper critical value (the largest variation coefficient) and 0=lcv<mcv<hcv; a determination unit, for determining whether lcnt/N is more than or equal to a first threshold, if yes, the variation degree for the set of respiratory rate data is low, if no, a further determination is made as to whether mcnt/N is more than or equal to the first threshold, if yes, the variation degree for the set of respiratory rate data is intermediate, if no, the variation degree for the set of respiratory rate data is high, where N is the total number of respiratory rate data in the set. The first threshold may be set to 70%, 80% or 90%, for example, as necessary.

Preferably, the base length calculation module comprises: a variation degree detection unit, for receiving the variation degree for the set of respiratory rate data from the variation degree calculation module and detecting the variation degree for the set of respiratory rate data; and a base length determination unit, for determining the base length for the set of respiratory rate data according to the detected variation degree for the set of respiratory rate data.

Preferably, the base length is equal to the number of reference respiratory rates in a predetermined first time period or the number of predetermined first respiratory rates, whichever is larger, when the variation degree for the set of respiratory rate data is low, the number of the predetermined first respiratory rates being the minimum empirical value of respiratory rates in the first time period; the base length is equal to the number of reference respiratory rates in a predetermined second time period or the number of predetermined second respiratory rates, whichever is larger, when the variation degree for the set of respiratory rate data is intermediate, the number of the predetermined second respiratory rates being the minimum empirical value of respiratory rates in the second time period; and the base length is equal to the number of reference respiratory rates in a predetermined third time period or the number of predetermined third respiratory rates, whichever is larger, when the variation degree for the set of respiratory rate data is high, the number of the predetermined third respiratory rates being the minimum empirical value of reference rates in the third time period; and wherein the reference respiratory rates represent the closest respiratory rates at present, the first time period<the second time period<the third time period, and the number of the first respiratory rates<the number of the second respiratory rates<the number of the third respiratory rates.

Preferably, the predetermined first time period may last 10 seconds, the number of the predetermined first respiratory rates may be 5, the predetermined second time period may last 15 seconds; the number of the predetermined second respiratory rates may be 6, the predetermined third time period may last 20 seconds; and the number of the predetermined third respiratory rates may be 7.

Preferably, the real-time degree calculation module comprises: a variation degree detection unit, for receiving the variation degree for the set of respiratory rate data from the variation degree calculation module and detecting the variation degree for the set of respiratory rate data; a weighting factor selection unit, for selecting a weighting factor according to the detected variation degree for the set of respiratory rate data, a higher variation degree leading to a smaller weighting factor; and a real-time degree calculation unit, for calculating the real-time degree for each of the set of

respiratory rate data according to

$$rt ( i ) = \frac{{i}^{n}}{{\sum }_{i = 1}^{i} {i}^{n}} ,$$

where i is the sequence number indicating the position for a data, the sequence number indicating the position for a preceding respiratory rate data is less than the sequence number indicating the position for a subsequent respiratory rate data, n is a weighting factor and n≧0, l represents the number of the respiratory rate data used in calculation, i.e. the weighted average length, and rt represents the real-time degree for the respiratory rate data.

Preferably, the weighting factor n=0 when the variation degree for the set of respiratory rate data is high; the weighting factor n=1 when the variation degree for the set of respiratory rate data is intermediate; and the weighting factor n=2 when the variation degree for the set of respiratory rate data is low.

Preferably, the weighted average respiratory rate is calculated by the weighted average respiratory rate calculation module according to:

$$rtav = {\sum }_{i = 1}^{t = sl} rr ( i ) * \frac{{i}^{n}}{{\sum }_{t = 1}^{sl} {i}^{n}}$$

where rtav represents the weighted average respiratory rate, i is the sequence number indicating the position for a data, the sequence number indicating the position for a preceding respiratory rate data is less than the sequence number indicating the position for a subsequent respiratory rate data, sl represents the weighted average length, n is a weighting factor and n≧0, and rr(i) represents the respiratory rate for the i^{th} data in the set of respiratory rate data.

The advantageous effects of the invention are embodied in that the calculated respiratory rate data are desirable in being both real time and smooth. The weighted averaging calculation result of the respiratory rate data is the weighted average of the respiratory rate data within a recent time period. By introducing the concept of the weighting factor in association with the variation of the real-time respiratory rates, the weighted average length and the latest real-time respiratory rates, the weighted averaging calculation result of the respiratory rate data may achieve better smoothness for real-time respiratory rate data with a higher variation degree, and better real-time degree for real-time respiratory rate data with a low variation degree.

Detailed descriptions will be made below to the features and advantages of the invention with reference to specific embodiments and accompanying drawings.

### BRIEF DESCRIPTION OF THE DRAWINGS

FIG. 1 is a flow chart showing a method for detecting respiratory rate according to an embodiment of the invention;

FIG. 2 is a diagram schematically simulating the calculation of the real-time degree according to the method for detecting respiratory rate shown in FIG. 1;

FIG. 3 is a diagram schematically simulating the calculation of the base length according to the method for detecting respiratory rate shown in FIG. 1;

FIG. 4 is a diagram schematically simulating the calculation of the weighted average length (with respect to the base length) according to the method for detecting respiratory rate shown in FIG. 1;

FIG. 5 is a diagram schematically simulating the calculation of the weighted average length (with respect to the reference respiratory rate) according to the method for detecting respiratory rate shown in FIG. 1;

FIG. 6 is a diagram schematically simulating the calculation of the weighted average respiratory rate according to the method for detecting respiratory rate shown in FIG. 1; and

FIG. 7 is a block diagram showing an apparatus for detecting respiratory rate according to an embodiment of the invention,

### DETAILED DESCRIPTION OF THE INVENTION

In the invention, the weighted averaging calculation result of the respiratory rate data is the weighted average of the respiratory rate data within a recent time period. It is of critical importance in the weighted averaging of the respiratory rates to set the time and weight to an optimal value so that the weighted average is calculated to approach and reflect the actual respiratory conditions to the most extent. The respiration of a human body shows a progressive process and sometimes may experience external interferences, which causes poorer signal quality and a drastic change in the calculated respiratory rates. Therefore, there is a need to weighted average the real-time respiratory rate data. The principle of the invention is to:

1. increase the stability and embody the real-time degree for signals with larger variations;

2. increase the real-time degree for signals with less variations and embody the change in the respiratory rates within a short time period;

3. employ more respiratory rate data to perform weighted averaging on signals with higher respiratory rates, so as to enhance the stability thereof; and

4. employ less respiratory rate data to perform weighted averaging on signals with lower respiratory rates, so as to enhance the real-time degree thereof.

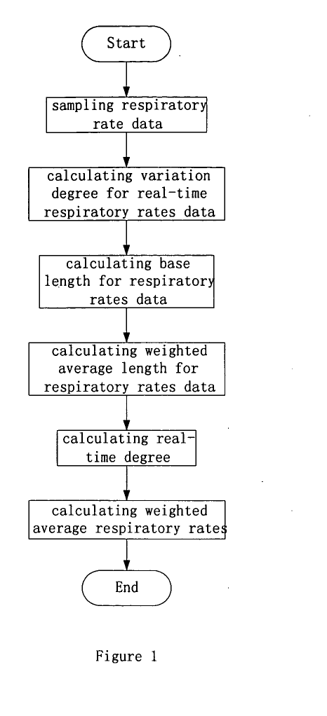

The inventive method for detecting respiratory rate mainly comprises the following steps in a sequence: sampling respiratory data; calculating the variation degree for the real-time respiratory rates; calculating the weighting factor; calculating the base length; calculating the weighted average length; and calculating the weighted average respiratory rate. FIG. 1 is a flow chart showing a method for detecting respiratory rate according to an embodiment of the invention, which comprises the following steps.

At step 1, the respiratory rate data received within a time period are sampled in a time sequence, to obtain a set of respiratory rate data, where the sequence number indicating the position for the first sampled respiratory rate data is the smallest, and a later sampled respiratory rate data will have a larger sequence number indicating its position.

At step 2, the variation coefficient and the variation degree for the set of respiratory rate data are calculated.

First, the variation coefficient for the set of respiratory rate data is calculated. The variation coefficient between two adjacent respiratory rate data is cv=(d1−d2)/((d1+d2)/2) if the values for the two adjacent respiratory rate data in the set of respiratory rate data are d1 and d2, respectively, where d1>=0, d2>=0 and d1 or d2 is not equal to 0.

The variation coefficient is a relative value, reflecting the change between two adjacent respiratory rate data.

If d1=d2, then cv=0, indicating there is no variation between two adjacent respiratory rate data;

If one d1 and d2 is equal to 0, then cv=2, indicating there is the biggest change between two adjacent respiratory rate data;

If d1 is more than d2, then cv>0, indicating d1 becomes higher with respective to d2 and the value of the variation coefficient cv reflects the change; and

If d1 is less than d2, then cv<0, indicating d1 becomes lower with respective to d2 and the value of the variation coefficient cv reflects the change.

Then, the variation degree for the set of respiratory rate data may be determined. Three critical values are provided: lcv is a lower critical value, mcv is an intermediate critical value, and hcv is an upper critical value (the maximum variation coefficient), where 0=<lcv<mcv<hcv=2, and the numbers lcnt, mcnt and hcnt of respiratory rate data whose variation coefficients fall within intervals [0, lcv], [0, mcv] and [0, hcv] are counted, respectively.

First, a determination is made as to whether lcnt/n>=80%. If yes, the variation degree s(n) is considered low. If no, a further determination is made as to whether mcnt/n>=80%. If yes, the variation degree s(n) is considered intermediate; if no, it may be determined that hcnt/n>=80% and the variation degree s(n) is then considered high. In practical applications, the above percentage 80% may be adjusted to other values as required.

In practical applications, we do not care whether the respiratory rate data becomes higher or lower, but only care the variation degree, which is the absolute value of the variation coefficient cv. For ease of calculation, cv may be amplified several times and then be rounded. At this time, cv may be given by:

cv=integer|2*M*(d1−d2)/(d1+d2)|,

where M is a scaling factor.

At step 3, the base length for the set of respiratory rate data is calculated.

Here, the base length represents the number of minimum respiratory rate data in these data involved in the calculation whose real-time degree is more than or equal to a predetermined value, which may be chosen according to particular requirements, for example, 70%, 80% or 90%. The reference respiratory rate represents the latest respiratory rate, having the highest real-time degree.

The calculation of the base length for the set of respiratory rate data comprises the following steps.

First, the variation degree for the set of respiratory rate data is detected; and then the base length for the set of respiratory rate data may be determined according to the variation degree for the set of respiratory rate data. The base length is equal to the number of reference respiratory rates in a predetermined first time period or the number of predetermined first respiratory rates, whichever is larger, when the variation degree for the set of respiratory rate data is low, the number of the predetermined first respiratory rates being the minimum empirical value of respiratory rates in the first time period; the base length is equal to the number of reference respiratory rates in a predetermined second time period or the number of predetermined second respiratory rates, whichever is larger, when the variation degree for the set of respiratory rate data is intermediate, the number of the predetermined second respiratory rates being the minimum empirical value of respiratory rates in the second time period; and the base length is equal to the number of reference respiratory rates in a predetermined third time period or the number of predetermined third respiratory rates, whichever is larger, when the variation degree for the set of respiratory rate data is high, the number of the predetermined third respiratory rates being the minimum empirical value of respiratory rates in the third time period; and wherein the reference respiratory rates represent the closest respiratory rates at present, the first time period<the second time period<the third time period, and the number of the first respiratory rates<the number of the second respiratory rates<the number of the third respiratory rates.

For example, when the variation degree for the set of respiratory rate data is low, the predetermined first time period may be set to 10 seconds, and the number of the predetermined first respiratory rates is 5, that is, the base length is the larger one of the number of reference respiratory rates in 10 seconds and 5, or MAX (5, the number of reference respiratory rates).

When the variation degree for the set of respiratory rate data is intermediate, the predetermined second time period may be set to 15 seconds, and the number of the predetermined first respiratory rates is 6, that is, the base length is the larger one of the number of reference respiratory rates in 15 seconds and 6, or MAX (6, the number of reference respiratory rates).

When the variation degree for the set of respiratory rate data is high, the predetermined third time period may be set to 20 seconds, and the number of the predetermined first respiratory rates is 7, that is, the base length is the larger one of the number of reference respiratory rates in 20 seconds and 7, or MAX (7, the number of reference respiratory rates).

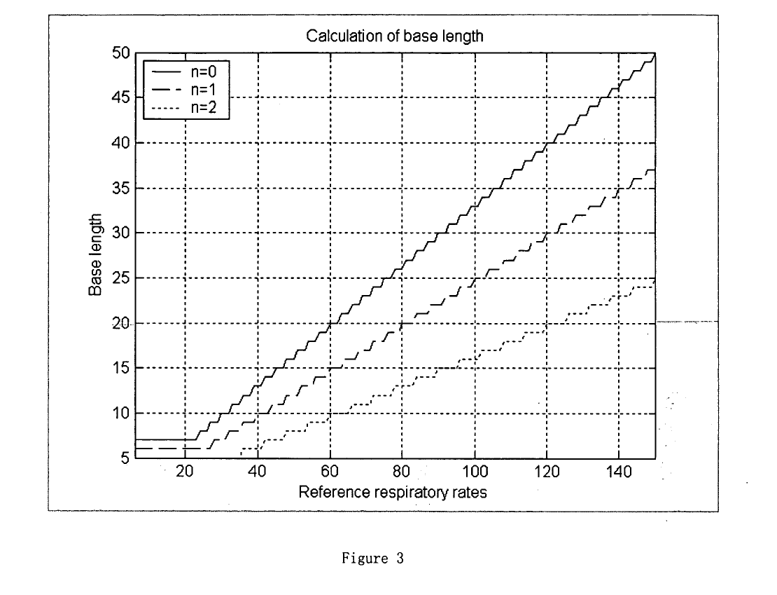

The relationship between the number of reference respiratory rates and the base length may be best depicted with a view in MATLAB, for example, as shown in FIG. 3, where n is a weighting factor.

From the calculation of the base length, it can be seen that the base length is related to the variation degree for the set of respiratory rate data, becoming higher with an increase in the number of reference respiratory rates. The higher the variation degree is, the faster the base length increases with an increase in the number of reference respiratory rates.

The predetermined first, second and third time period may have different values according to particular requirements. A longer time leads to a slower change and a smaller number of respiratory rates; a shorter time leads to a faster change and a larger number of respiratory rates.

At step 4, the weighted average length for the set of respiratory rate data is calculated.

The weighted average length is the number of respiratory rate data used in calculation of the real-time average of the respiratory rates.

It is defined that the relationship between the weighted average length and the base length is given by the following equation:

$$\frac{blrt}{slrt} = \frac{{\sum }_{t = sl - bl + 1}^{sl} {i}^{n}}{{\sum }_{i = 1}^{sl} {i}^{n}} >= 90 \%$$

where i is the sequence number indicating the position for a data, n is a weighting factor and n≧0, b1 represents the base length, s1 represents the weighted average length, which is the least integer satisfying the equation. The above percentage 90% may be set to other values as required.

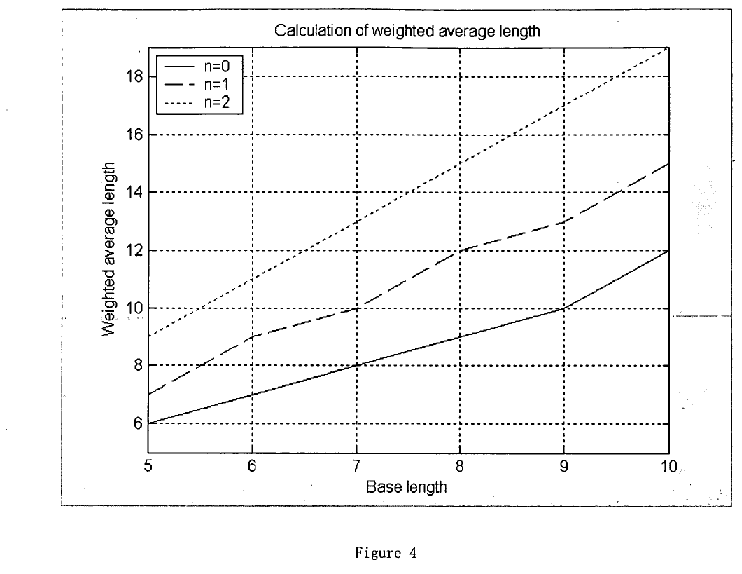

Once the base length for the set of respiratory rate data is determined, the weighted average length for the set of respiratory rate data may be calculated. The relationship between the weighted average length and the base length is best depicted with a view in MATLAB, for example, as shown in FIG. 4, where n is a weighting factor.

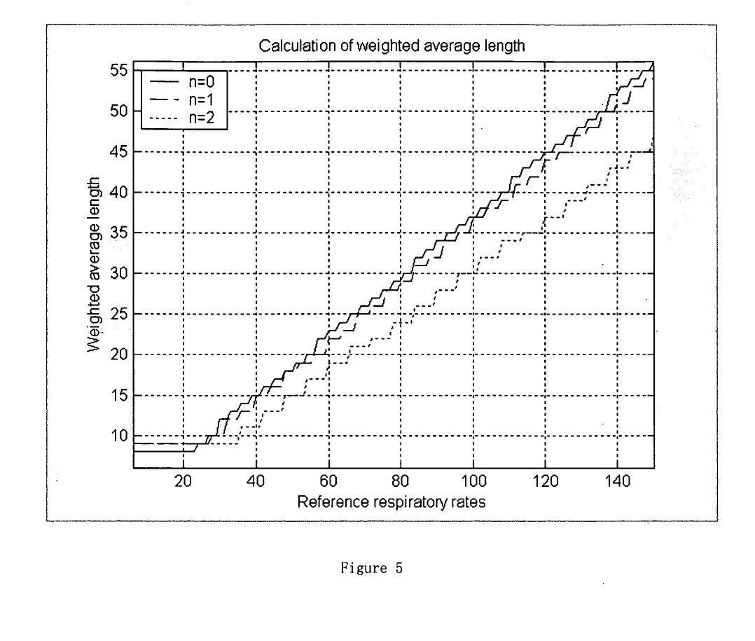

The relationship between the number of reference respiratory rates and the weighted average length is best depicted with a view in MATLAB, for example, as shown in FIG. 5, where n is a weighting factor.

At step 5, the real-time degree and the weighting factor for each of the set of respiratory rate data are calculated.

First, the variation degree for the set of respiratory rate data is detected.

Then, the weighting factor n is determined according to a basic rule that a higher variation degree leads to a smaller weighting factor. For example, the weighting factor n is determined according to the following rule: n=0 when the variation degree for the sampled set of respiratory rate data is high, n=1 when the variation degree for the sampled set of respiratory rate data is intermediate, and n=2 when the variation degree for the sampled set of respiratory rate data is low.

In the last, the real-time degree for the set of respiratory rate data is calculated.

The real-time degree specifies the real-time degree for a respiratory rate data, that is, the closeness between the respiratory rate data and the current respiratory rate data. A higher real-time degree indicates that the respiratory rate data has a better real-time degree. It may be denoted as the weighting factor for the respiratory rate data, that is

$$rt ( i ) = \frac{{i}^{n}}{{\sum }_{i = 1}^{i} {i}^{n}} ,$$

where rt represents the real-time degree, i is the sequence number indicating the position for a data, the sequence number indicating the position for a preceding respiratory rate data is less than the sequence number indicating the position for a subsequent respiratory rate data, n is a weighting factor and n≧0, and l represents the weighted average length calculated based on the base length.

Taking the last 10 respiratory rate data as an example. Table 1 lists the relationship between the real-time degree and the weighting factor below.

TABLE 1

| | Data position sequence number | | | | | | | | | |
|---|---|---|---|---|---|---|---|---|---|---|
| | 1 | 2 | 3 | 4 | 5 | 6 | 7 | 8 | 9 | 10 |
| Data generation time | —————————————> | | | | | | | | | |
| direction | | | | Past | | | Now | | | |
| Weighted value | 1 | 1 | 1 | 1 | 1 | 1 | 1 | 1 | 1 | 1 |
| (weighting factor = 0) | | | | | | | | | | |
| Real-time degree | 0.1 | 0.1 | 0.1 | 0.1 | 0.1 | 0.1 | 0.1 | 0.1 | 0.1 | 0.1 |
| (weight coefficient) | | | | | | | | | | |
| Weighted value | 1 | 2 | 3 | 4 | 5 | 6 | 7 | 8 | 9 | 10 |
| (weighting factor = 1) | | | | | | | | | | |
| Real-time degree | 0.018 | 0.036 | 0.054 | 0.072 | 0.09 | 0.109 | 0.127 | 0.145 | 0.163 | 0.181 |
| (weight coefficient) | | | | | | | | | | |
| Weighted value | 1 | 4 | 9 | 16 | 25 | 36 | 49 | 64 | 81 | 100 |
| (weighting factor = 2) | | | | | | | | | | |
| Real-time degree | 0.002 | 0.01 | 0.023 | 0.041 | 0.064 | 0.093 | 0.127 | 0.166 | 0.21 | 0.259 |
| (weight coefficient) | | | | | | | | | | |

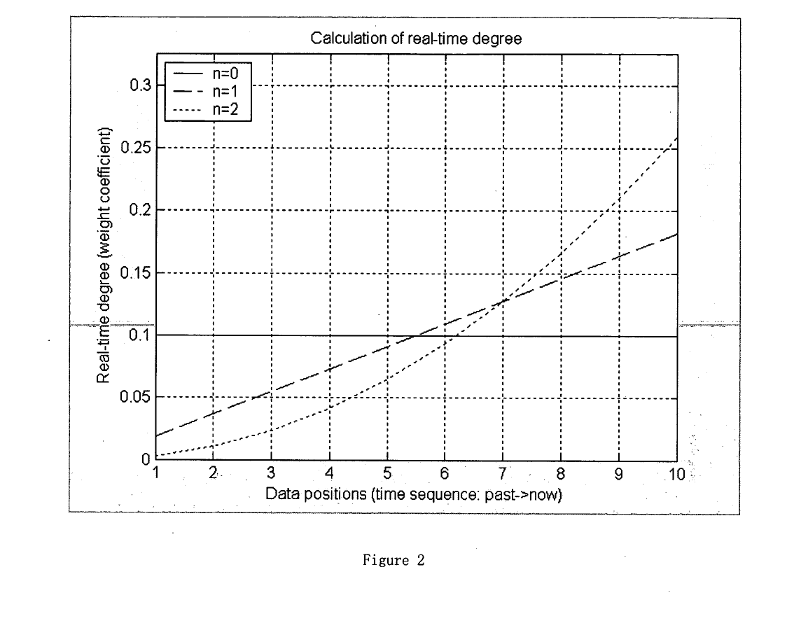

The above table may be best depicted with a view in MATLAB, for example, as shown in FIG. 2, where n is a weighting factor.

It may be seen from the calculation of the real-time degree that the real-time degree for a respiratory rate data is related to the weighting factor n for the set of respiratory rate data and the sequence number indicating the position of the respiratory rate data (the position in the set of respiratory rate data). From the determination of the weighting factor n and the sequence number indicating the position of the respiratory rate data, it may be known that the sequence number indicating the position for a preceding respiratory rate data is less than the sequence number indicating the position for a subsequent respiratory rate data, and a larger variation degree leads to a smaller weighting factor n so that a mapping relationship occurs between the real-time degree and the variation degree. This relationship may be indicated with the weighting factor. For data with a larger variation degree, a smaller weighting factor is selected and the data at different time sequence has a smaller difference in real-time degrees, so that the calculated respiratory rate has an increased stability and the stability is enhanced for data with a larger variation degree. For data with a smaller variation degree, a larger weighting factor is selected and the data at different time sequence has a larger difference in real-time degrees (data at a closer time has a higher real-time degree), so that the calculated respiratory rate has an increased real-time degree and the real-time degree is enhanced for data with a smaller variation degree.

At step 6, the weighted average respiratory rate for the set of respiratory rate data is calculated.

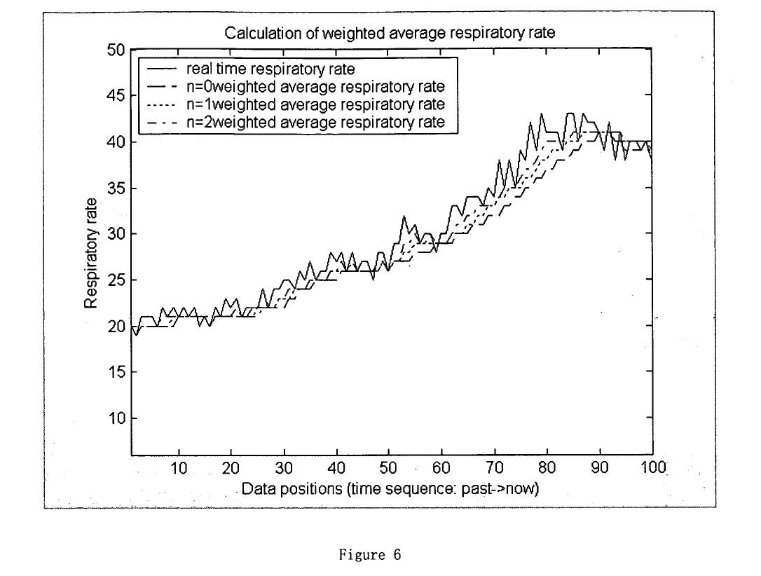

The weighted average respiratory rate may be derived from:

$$rtav = {\sum }_{i = 1}^{t = sl} rr ( i ) * \frac{{i}^{n}}{{\sum }_{t = 1}^{sl} {i}^{n}}$$

where rtav represents the weighted average respiratory rate, i is the sequence number indicating the position for a data, the sequence number indicating the position for a preceding respiratory rate data is less than the sequence number indicating the position for a subsequent respiratory rate data, n is a weighting factor and n>0, and rr(i) represents the respiratory rate for the i^{th} data in the set of respiratory rate data. The calculation of the weighted average respiratory rate may be best depicted with a view in MATLAB, for example, as shown in FIG. 6, where n is a weighting factor.

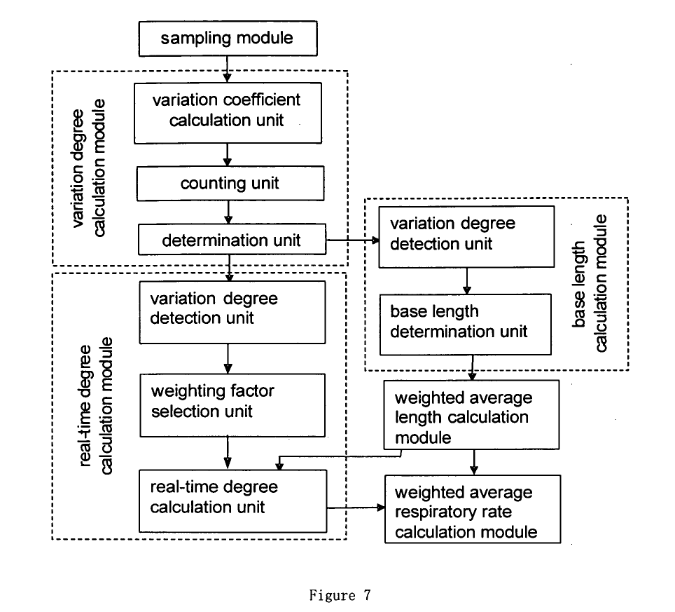

FIG. 7 shows an apparatus for detecting respiratory rate to implement the above method according to an embodiment of the invention, comprising: a sampling module, a variation degree calculation module, a real-time degree calculation module, a base length calculation module, a weighted average length calculation module, and a weighted average respiratory rate calculation module.

The sampling module is configured to sample a set of respiratory rate data in a time sequence.

The variation degree calculation module is configured to receive the set of respiratory rate data from the sampling module and determine the variation degree for the set of respiratory rate data. The variation degree calculation module comprises a variation coefficient calculation unit, a counting unit and a determination unit, which are connected with each other in said order. The variation coefficient calculation unit is configured to receive the set of respiratory rate data from the sampling module and calculate the variation coefficient between two adjacent respiratory rate data in the set of respiratory rate data, wherein the variation coefficient between two adjacent respiratory rate data in the set of respiratory rate data is cv=(d1−d2)/((d1+d2)/2) if the values for the two adjacent respiratory rate data are d1 and d2, respectively, where d1>=0, d2>=0 and d1 or d2 is not equal to 0. The counting unit is configured to count the numbers lcnt, mcnt and hcnt of respiratory rate data whose variation coefficients fall within intervals [0, lcv], [0, mcv] and [0, hcv] respectively, where lcv is a lower critical value, mcv is an intermediate critical value, hcv is an upper critical value (the largest variation coefficient) and 0=<lcv<mcv<hcv. The determination unit is configured to determine whether lcnt/N is more than or equal to a first threshold, if yes, the variation degree for the set of respiratory rate data is low, if no, a further determination is made as to whether mcnt/N is more than or equal to the first threshold, if yes, the variation degree for the set of respiratory rate data is intermediate, if no, the variation degree for the set of respiratory rate data is high, where N is the total number of respiratory rate data in the set. The first threshold may be set as required, to 70%, 80% or 90% for example.

The base length calculation module is configured to receive the variation degree for the set of respiratory rate data from the determination unit in the variation degree calculation module and calculate the base length for the set of respiratory rate data according to the determined variation degree for the set of respiratory rate data. The base length calculation module comprises a variation degree detection unit and a base length determination unit connected with each other. The variation degree detection unit is configured to receive and detect the variation degree for the set of respiratory rate data from the determination unit of the variation degree calculation module. The base length determination unit is configured to determine the base length for the set of respiratory rate data according to the detected variation degree for the set of respiratory rate data. The base length is equal to the number of reference respiratory rates in a predetermined first time period or the number of predetermined first respiratory rates, whichever is larger, when the variation degree for the set of respiratory rate data is low, the number of the predetermined first respiratory rates being the minimum empirical value of respiratory rates in the first time period; the base length is equal to the number of reference respiratory rates in a predetermined second time period or the number of predetermined second respiratory rates, whichever is larger, when the variation degree for the set of respiratory rate data is intermediate, the number of the predetermined second respiratory rates being the minimum empirical value of respiratory rates in the second time period; and the base length is equal to the number of reference respiratory rates in a predetermined third time period or the number of predetermined third respiratory rates, whichever is larger, when the variation degree for the set of respiratory rate data is high, the number of the predetermined third respiratory rates being the minimum empirical value of respiratory rates in the third time period; and wherein the reference respiratory rates represent the closest respiratory rates at present, the first time period<the second time period<the third time period, and the number of the first respiratory rates<the number of the second respiratory rates<the number of the third respiratory rates. In the embodiment, the predetermined first time period lasts 10 seconds, the number of the predetermined first respiratory rates is 5, the predetermined second time period lasts 15 seconds, the number of the predetermined second respiratory rates is 6, the predetermined third time period lasts 20 seconds, and the number of the predetermined third respiratory rates is 7.

The weighted average length calculation module is configured to receive the base length for the set of respiratory rate date from the base length calculation module and calculate the weighted average length for the set of respiratory rate data based on the determined base length.

The real-time degree calculation module is configured to receive the variation degree for the set of respiratory rate data from the variation degree calculation module and the weighted average length for the set of respiratory rate data from the weighted average length calculation module, and calculate the real-time degree for each of the set of respiratory rate data according to the variation degree for the set of respiratory rate data, the sequence number indicating the position for each of the set of respiratory rate date and the weighted average length, the sequence number indicating the position for a preceding respiratory rate data is less than the sequence number indicating the position for a subsequent respiratory rate data. The real-time degree calculation module comprises a variation degree detection unit, a weighting factor selection unit and a real-time degree calculation unit, which are connected with each other in said order. The variation degree detection unit is used for receiving and detecting the variation degree for the set of respiratory rate data from the variation degree calculation module. The weighting factor selection unit is used for selecting a weighting factor according to the detected variation degree for the set of respiratory rate data, a higher variation degree leading to a smaller weighting factor. The real-time degree calculation unit is used for calculating the real-time degree for each of the set of respiratory rate data according to

$$rt ( i ) = \frac{{i}^{n}}{{\sum }_{i = 1}^{i} {i}^{n}} ,$$

where i is the sequence number indicating the position for a data, the sequence number indicating the position for a preceding respiratory rate data is less than the sequence number indicating the position for a subsequent respiratory rate data, n is a weighting factor and n≧0, l represents the number of the respiratory rate data used in the calculation, i.e. the weighted average length, and rt represents the real-time degree for the respiratory rate date. The weighting factor n=0 when the variation degree for the set of respiratory rate data is high, the weighting factor n=1 when the variation degree for the set of respiratory rate data is intermediate, and the weighting factor n=2 when the variation degree for the set of respiratory rate data is low.

The weighted average respiratory rate calculation module is used for receiving the weighted average length from the weighted average length calculation module and the real-time degree from the real-time degree calculation module, and calculating the weighted average respiratory rate for the set of respiratory rate data according to the weighted average length and the real-time degree. The weighted average respiratory rate calculation module calculates the weighted average respiratory by:

$$rtav = {\sum }_{i = 1}^{t = sl} rr ( i ) * \frac{{i}^{n}}{{\sum }_{t = 1}^{sl} {i}^{n}}$$

where rtav represents the weighted average respiratory rate, l is the sequence number indicating the position for a data, the sequence number indicating the position for a preceding respiratory rate data is less than the sequence number indicating the position for a subsequent respiratory rate data, sl represents the weighted average length, n is a weighting factor and n≧0, and rr(i) represents the respiratory rate for the i^{th} data in the set of respiratory rate data.

According to the method and apparatus for detecting respiratory rate of the invention, a smaller weighting factor end a larger base length are used for the signals with a larger variation to enhance the stability of the signals and embody the real-time degree of the respiratory rates as well, while a larger weighting factor and a smaller base length are used for signals with a smaller variation to enhance the real-time degree and embody the change of the respiratory rates in a short time. For the signals with a higher reference respiratory rate (that is, the reference respiratory period thereof is smaller), more respiratory rate data are used for the weighted averaging to enhance the stability of the signals, while for the signals with a lower reference respiratory rate (that is, the reference respiratory period thereof is larger), less respiratory rate data are used for the weighted averaging to reflect the real-time degree of the signals. Practically, a better effect for the weighted average respiratory rate may be trade off between the real-time degree and the smoothness by selecting the weighting factor and determining the base length.

It is to be understood by those skilled in the art that the specific embodiments in the invention are intended to be illustrative rather than limiting. Various improvements and modifications may be made without departing from the spirit and features of the invention, the scope of which is to be defined by the attached claims, rather than the description.

What is claimed is:

1. A method for detecting respiratory rate, comprising:
A1. sampling a set of respiratory rate data in a time sequence;
B1. determining the variation degree for the set of respiratory rate data;
C1. determining the base length for the set of respiratory rate data according to the variation degree for the set of respiratory rate data;
D1. calculating the weighted average length for the set of respiratory rate data according to the base length for the set of respiratory rate data;
E1. calculating the real-time degree for each of the set of respiratory rate data according to the variation degree for the set of respiratory rate data, the sequence number indicating the position for each of the set of respiratory rate data and the weighted average length for the set of respiratory rate data, the sequence number indicating the position for a preceding respiratory rate data is less than the sequence number indicating the position for a subsequent respiratory rate data; and
F1. calculating the weighted average respiratory rate according to the weighted average length and the real-time degree.

2. The method for detecting respiratory rate as set forth in claim 1, wherein the step B1 comprises the steps of:
B11. calculating the variation coefficient between two adjacent respiratory rate data in the set of respiratory rate data;
B12. counting the numbers lcnt, mcnt and hcnt of respiratory rate data whose variation coefficients fall within intervals [0, lcv], [0, mcv] and [0, hcv], respectively, where lcv is a lower critical value, mcv is an intermediate critical value, hcv is an upper critical value (the maximum variation coefficient) and 0=<lcv<mcv<hcv;
B13. determining whether lcnt/N is more than or equal to a first threshold. If yes, the variation degree for the set of respiratory rate data is low, if no, the step B14 is performed, where N is the total number of respiratory rate data in the set;
B14. determining whether mcnt/N is more than or equal to the first threshold, if yes, the variation degree for the set of respiratory rate data is intermediate, if no, the step B15 is performed; and
B15. determining that the variation degree for the set of respiratory rate data is high.

3. The method for detecting respiratory rate as set forth in claim 2, wherein the variation coefficient between two adjacent respiratory rate data is cv=(d1−d2)/((d1+d2)/2) if the values for the two adjacent respiratory rate data in the set of respiratory rate data are d1 and d2, respectively, where d1>=0, d2>=0 and d1 and d2 are not equal to 0 at the same time (d1 or d2 is not equal to 0).

4. The method for detecting respiratory rate as set forth in claim 3, wherein the variation coefficient between the two adjacent respiratory rate data is cv=integer|2*M*(d1−d2)/((d1+d2)|, where M is a scaling factor.

5. The method for detecting respiratory rate as set forth in claim 2, wherein the first threshold is 70%, 80% or 90%.

6. The method for detecting respiratory rate as set forth in claim 1, wherein the step C1 comprises the steps of:
C11. detecting the variation degree for the set of respiratory rate data; and
C12. determining the base length for the set of respiratory rate data according to the detected variation degree for the set of respiratory rate data, wherein the base length is equal to the number of reference respiratory rates in a predetermined first time period or the number of predetermined first respiratory rates, whichever is larger, when the variation degree for the set of respiratory rate data is low, the number of the predetermined first respiratory rates being the minimum empirical value of respiratory rates in the first time period;
the base length is equal to the number of reference respiratory rates in a predetermined second time period or the number of predetermined second respiratory rates, whichever is larger, when the variation degree for the set of respiratory rate data is intermediate, the number of the predetermined second respiratory rates being the minimum empirical value of respiratory rates in the second time period; and
the base length is equal to the number of reference respiratory rates in a predetermined third time period or the number of predetermined third respiratory rates, whichever is larger, when the variation degree for the set of respiratory rate data is high, the number of the predetermined third respiratory rates being the minimum empirical value of respiratory rates in the third time period;
wherein the reference respiratory rates represent the closest respiratory rates at present, the first time periods the second time period<the third time period, and the number of the first respiratory rates<the number of the second respiratory rates<the number of the third respiratory rates.

7. The method for detecting respiratory rate as set forth in claim 6, wherein the predetermined first time period lasts 10 seconds, the number of the predetermined first respiratory rates is 5, the predetermined second time period lasts 15 seconds, the number of the predetermined second respiratory rates is 6, the predetermined third time period lasts 20 seconds, and the number of the predetermined third respiratory rates is 7.

8. The method for detecting respiratory rate as set forth in claim 1, wherein the weighted average length is calculated by the following equation at the step of D1,
$$\frac{blrt}{slrt} = \frac{{\sum }_{t = sl - bl + 1}^{sl} {i}^{n}}{{\sum }_{i = 1}^{sl} {i}^{n}} >= 90 \%$$
where l is the sequence number indicating the position for a data, the sequence number indicating the position for a preceding respiratory rate data is less than the sequence number indicating the position for a subsequent respiratory rate data, n is a weighting factor and n≧0, b1 represents the base length, s1 represents the weighted average length, which is the least integer satisfying the equation.

9. The method for detecting respiratory rate as set forth in claim 8, wherein the weighting factor n=0 when the variation degree for the set of respiratory rate data is high;
the weighting factor n=1 when the variation degree for the set of respiratory rate data is intermediate; and
the weighting factor n=2 when the variation degree for the set of respiratory rate data is low.

10. The method for detecting respiratory rate as set forth in claim 1, wherein the step E1 comprises the steps of:
E11. detecting the variation degree for the set of respiratory rate data;
E12. selecting a weighting factor based on the variation degree for the set of respiratory rate data, a higher variation degree leading to a smaller weighting factor; and
E13. calculating the real-time degree for each of the set of respiratory rate data according to
$$rt ( i ) = \frac{{i}^{n}}{{\sum }_{i = 1}^{i} {i}^{n}} ,$$
where i is the sequence number indicating the position for a data, the sequence number indicating the position for a preceding respiratory rate data is less than the sequence number indicating the position for a subsequent respiratory rate data, n is a weighting factor and n≧0, l represents the weighted average length for the data used, and rt represents the real-time degree.

11. The method for detecting respiratory rate as set forth in claim 10, wherein the weighting factor n=0 when the variation degree for the set of respiratory rate data is high;
the weighting factor n=1 when the variation degree for the set of respiratory rate data is intermediate; and
the weighting factor n=2 when the variation degree for the set of respiratory rate data is low.

12. The method for detecting respiratory rate as set forth in claim 1, wherein the weighted average respiratory rate is calculated at the step F1 by
$$rtav = {\sum }_{i = 1}^{t = sl} rr ( i ) * \frac{{i}^{n}}{{\sum }_{t = 1}^{sl} {i}^{n}}$$
where rtav represents the weighted average respiratory rate, i is the sequence number indicating the position for a data, the sequence number indicating the position for a preceding respiratory rate data is less than the sequence number indicating the position for a subsequent respiratory rate data, sl represents the weighted average length, n is a weighting factor and n≧0, and rr(l) represents the respiratory rate for the i^{th} data in the set of respiratory rate data.

13. The method for detecting respiratory rate as set forth in claim 12, wherein the weighting factor n=0 when the variation degree for the set of respiratory rate data is high;
the weighting factor n=1 when the variation degree for the set of respiratory rate data is intermediate; and
the weighting factor n=2 when the variation degree for the set of respiratory rate data is low.

14. An apparatus for detecting respiratory rate, comprising:
a sampling module, for sampling a set of respiratory rate data in a time sequence;
a variation degree calculation module, for receiving the set of respiratory rate data from the sampling module and determining the variation degree for the set of respiratory rate data;
a base length calculation module, for calculating the base length for the set of respiratory rate data according to the determined variation degree for the set of respiratory rate data;
a weighted average length calculation module, for calculating the weighted average length for the set of respiratory rate data according to the determined base length for the set of respiratory rate data;
a real-time degree calculation module, for calculating the real-time degree for each of the set of respiratory rate data according to the variation degree for the set of respiratory rate data from the variation degree calculation module, the sequence number indicating the position for each of the set of respiratory rate data and the weighted average length for the set of respiratory rate data from the weighted average length calculation module, the sequence number indicating the position for a preceding respiratory rate data is less than the sequence number indicating the position for a subsequent respiratory rate data; and
a weighted average respiratory rate calculation module, for calculating the weighted average respiratory rate for the set of respiratory rate data according to the calculated weighted average length and the calculated real-time degree.

15. The apparatus for detecting respiratory rate as set forth in claim 14, wherein the variation degree calculation module comprises:
a variation coefficient calculation unit, for receiving the set of respiratory rate data from the sampling module and calculating the variation coefficient between two adjacent respiratory rate data in the set of respiratory rate data;
a counting unit, for counting the numbers lcnt, mcnt and hcnt of respiratory rate data whose variation coefficients fall within intervals [0, lcv], [0, mcv] and [0, hcv] respectively, where lcv is a lower critical value, mcv is an intermediate critical value, hcv is an upper critical value (the largest variation coefficient) and 0=<lcv<mcv<hcv;
a determination unit, for determining whether lcnt/N is more than or equal to a first threshold, if yes, the variation degree for the set of respiratory rate data is low, if no, a further determination is made as to whether mcnt/N is more than or equal to the first threshold, if yes, the variation degree for the set of respiratory rate data is intermediate, if no, the variation degree for the set of respiratory rate data is high, where N is the total number of respiratory rate data in the set.

16. The apparatus for detecting respiratory rate as set forth in claim 15, wherein the variation coefficient between two adjacent respiratory rate data is cv=(d1−d2)/((d1+d2)/2) if the values for the two adjacent respiratory rate data in the set of respiratory rate data are d1 and d2, respectively, where d1>=0, d2>=0 and d1 and d2 are not equal to 0 at the same time (d1 or d2 is not equal to 0).

17. The apparatus for detecting respiratory rate as set forth in claim 16, wherein the variation coefficient between the two adjacent respiratory rate data is cv=integer|2*M*(d1−d2)/((d1+d2)|, where M is a scaling factor.

18. The apparatus for detecting respiratory rate as set forth in claim 15, wherein the first threshold is 70%, 80% or 90%.

19. The apparatus for detecting respiratory rate as set forth in claim 14, wherein the base length calculation module comprises:
a variation degree detection unit, for receiving the variation degree for the set of respiratory rate data from the variation degree calculation module and detecting the variation degree for the set of respiratory rate data; and
a base length determination unit, for determining the base length for the set of respiratory rate data according to the detected variation degree for the set of respiratory rate data.

20. The apparatus for detecting respiratory rate as set forth in claim 19, wherein the base length is equal to the number of reference respiratory rates in a predetermined first time period or the number of predetermined first respiratory rates, whichever is larger, when the variation degree for the set of respiratory rate data is low, the number of the predetermined first respiratory rates being the minimum empirical value of respiratory rates in the first time period;
the base length is equal to the number of reference respiratory rates in a predetermined second time period or the number of predetermined second respiratory rates, whichever is larger, when the variation degree for the set of respiratory rate data is intermediate, the number of the predetermined second respiratory rates being the minimum empirical value of respiratory rates in the second time period; and
the base length is equal to the number of reference respiratory rates in a predetermined third time period or the number of predetermined third respiratory rates, whichever is larger, when the variation degree for the set of respiratory rate data is high, the number of the predetermined third respiratory rates being the minimum empirical value of respiratory rates in the third time period;
wherein the reference respiratory rates represent the closest respiratory rates at present, the first time periods the second time period<the third time period, and the number of the first respiratory rates<the number of the second respiratory rates<the number of the third respiratory rates.

21. The apparatus for detecting respiratory rate as set forth in claim 20, wherein the predetermined first time period lasts 10 seconds, the number of the predetermined first respiratory rates is 5;
the predetermined second time period lasts 15 seconds, the number of the predetermined second respiratory rates is 6; and
the predetermined third time period lasts 20 seconds, and the number of the predetermined third respiratory rates is 7.

22. The apparatus for detecting respiratory rate as set forth in claim 14, wherein the real-time degree calculation module comprises:
a variation degree detection unit, for receiving the variation degree for the set of respiratory rate data from the variation degree calculation module and detecting the variation degree for the set of respiratory rate data;
a weighting factor selection unit, for selecting a weighting factor according to the detected variation degree for the set of respiratory rate data, a higher variation degree leading to a smaller weighting factor; and
a real-time degree calculation unit, for calculating the real-time degree for each of the set of respiratory rate data according to
$$rt ( i ) = \frac{{i}^{n}}{{\sum }_{i = 1}^{i} {i}^{n}} ,$$
where i is the sequence number indicating the position for a data, the sequence number indicating the position for a preceding respiratory rate data is less than the sequence number indicating the position for a subsequent respiratory rate data, n is a weighting factor and n≧0, l represents the weighted average length for the data used, and rt represents the real-time degree.

23. The apparatus for detecting respiratory rate as set forth in claim 22, wherein the weighting factor n=0 when the variation degree for the set of respiratory rate data is high;
the weighting factor n=1 when the variation degree for the set of respiratory rate data is intermediate; and
the weighting factor n=2 when the variation degree for the set of respiratory rate data is low.

24. The apparatus for detecting respiratory rate as set forth in claim 14, wherein the weighted average respiratory rate is calculated by the weighted average respiratory rate calculation module according to:
$$rtav = {\sum }_{i = 1}^{t = sl} rr ( i ) * \frac{{i}^{n}}{{\sum }_{t = 1}^{sl} {i}^{n}}$$
where rtav represents the weighted average respiratory rate, i is the sequence number indicating the position for a data, the sequence number indicating the position for a preceding respiratory rate data is less than the sequence number indicating the position for a subsequent respiratory rate data, sl represents the weighted average length, n is a weighting factor and n≧0, and rr(i) represents the respiratory rate for the i^{th} data in the set of respiratory rate data.
